(19) United States
(12) Patent Application Publication
(10) Pub. No.: US 2021/0267777 A1
(43) Pub. Date: Sep. 2, 2021

(54) ORTHOPEDIC DEVICE HAVING A FOOT PART, A LOWER-LEG PART AND A THIGH PART

(71) Applicant: OTTOBOCK SE & CO. KGAA, Duderstadt (DE)

(72) Inventors: Malte BELLMANN, Hann. Münden (DE); Herman BOITEN, Ede (NL); Paul WEBER, Wiesbaden (DE)

(73) Assignee: OTTOBOCK SE & CO. KGAA, Duderstadt (DE)

(21) Appl. No.: 17/260,536

(22) PCT Filed: Jul. 3, 2019
(86) PCT No.: PCT/EP2019/067887

§ 371 Date: Jan. 14, 2021

(30) Foreign Application Priority Data
Jul. 18, 2018 (DE) 10 2018 117 377.6

(51) Int. Cl. A61F 2/68; A61F 2/66; A61F 2/64

(52) U.S. Cl. CPC A61F 2/68; A61F 2/6607; A61F 2002/745; A61F 2002/6614; A61F 2/64

(57) ABSTRACT

An orthopedic device that includes a foot part, a lower-leg part, and a thigh part. The foot part is connected to the lower-leg part for pivoting about a first pivot axis by an ankle joint. The lower-leg part is connected to the thigh part for pivoting about a second pivot axis by a knee joint. The foot part is connected to the thigh part by a force-transmitting apparatus. The force-transmitting apparatus causes a dorsal flexion of the foot part in the event of a knee flexion over a first knee flexion angle range and causes a plantar flexion of the foot part in the event of further knee flexion after a knee flexion limit angle has been exceeded.

The invention relates to an orthopedic device having a foot part, a lower-leg part and a thigh part, the foot part being connected by an ankle joint to the lower-leg part so as to be pivotable about a first pivot axis, and the lower-leg part being connected by a knee joint to the thigh part so as to be pivotable about a second pivot axis. The foot part is additionally connected to the thigh part by a force-transmitting mechanism. Such an orthopedic device is embodied in particular as a prosthesis or an orthosis. In an embodiment as a prosthesis, the thigh part is preferably designed as a thigh socket, for receiving a stump, or as a prosthetic knee-joint upper part that is connectable to such a thigh socket. In an embodiment of an orthopedic device as an orthosis, at least one securing mechanism is arranged on the thigh part and allows the thigh part, for example a thigh rail or a thigh shell, to be placed on a patient's thigh.

The purpose of orthoses is to guide or support the movement of an existing limb or to brace and support a limb. Orthoses for the lower limb are available in different designs. Those referred to as knee ankle foot orthoses (KAFO) support both the foot and also the ankle joint and knee joint. The foot is generally placed on a foot plate, one or more lower-leg rails extend parallel to the lower leg, and an orthotic knee joint is provided approximately in the region of the natural knee axis. Securing devices are mounted on one or more thigh rails in order to fasten the orthosis to the thigh. Likewise, securing devices can be provided on the lower-leg part or on the foot plate, so as to be able to fasten the orthosis to the respective leg that is to be managed.

Prostheses with a prosthetic knee joint have, as the foot part, a prosthetic foot which is connected to the prosthetic knee joint via a lower-leg tube serving as lower-leg part. Proximally with respect to the prosthetic knee joint axis, a securing device is provided for the prosthesis, so as to fasten the prosthesis to the thigh stump. Various types of prosthetic knee joints can be used, for example monoaxial prosthetic knee joints, polycentric knee joints with damping mechanisms, or computer-controlled and driven active prosthetic knee joints.

DE 10 2012 023 023 A1 discloses an orthopedic device for orthotic or prosthetic management of a patient, said device comprising a knee joint which has a proximal upper part, and a distal lower part arranged on the latter in such a way as to be pivotable about a knee axis. An ankle joint is also provided, which has an ankle joint axis, and a foot part which is arranged distally on the ankle joint and is pivotable about the ankle joint axis. A lower-leg part is arranged between the ankle joint and the knee joint. To make available a coupling between the knee joint and the ankle joint with the least possible outlay in terms of design and to allow the kinetic energy of the knee to be used for an ankle movement, such that an approximation to the natural gait pattern is afforded, the upper part of the knee joint is coupled to the foot part by a force-transmitting mechanism in which a plantar flexion of the foot part is brought about in the event of a knee flexion. In this way, at the end of the stance phase, when a knee flexion starts, a plantar flexion is performed in order to lengthen the leg length during the bending of the knee. In this way, the duration of the ground contact of the foot part is prolonged and the vertical movement of the center of gravity of the body is minimized.

US 2008/0269913 A1 discloses an artificial leg with a prosthetic knee joint and a prosthetic foot. On the prosthetic knee joint, a connection rod is secured frontally with respect to the knee joint axis, such that, upon flexion of the knee, the connection rod is moved in a guide in the lower leg. The movement is conveyed to the prosthetic foot via a tensioning element, such that the tip of the foot is lifted in the event of a flexion of the knee.

EP 0 041 052 B1 relates to a prosthesis for a lower limb, in which a thigh socket and a lower leg are coupled to each other via a toothed hinge. A spring-loaded piston rod lifts the toes in the event of a flexion of the knee.

DE 47 53 03 B1 relates to an artificial leg in which a lower-leg part and a thigh part are connected to each other by two articulated rods, in order to cause dorsiflexion when the prosthetic knee joint is placed at an angle.

The coupling of a dorsiflexion with a knee flexion is effected in order to facilitate the swing-through of an artificial leg. If the foot tip in the context of a dorsiflexion is not lifted during the swing phase, there is the danger of the foot tip trailing on the ground and becoming caught. This is often compensated by a unnatural gait pattern in which circumduction takes place.

Users of an orthopedic device for a lower limb not only face challenges when walking but also when seated, when sitting down and when standing up, because the function of the muscles is limited or lost in those persons using orthopedic devices for the lower limb.

The object of the present invention is to make available an orthopedic device that makes it easier to sit down and stand up.

According to the invention, this object is achieved by a device having the features of the main claim. Advantageous embodiments and developments of the invention are disclosed in the dependent claims, the description and the figures.

In the orthopedic device according to the invention, having a foot part, a lower-leg part and a thigh part, the foot part being connected by an ankle joint to the lower-leg part so as to be pivotable about a first pivot axis, the lower-leg part being connected by a knee joint to the thigh part so as to be pivotable about a second pivot axis, and the foot part being connected to the thigh part by a force-transmitting mechanism, provision is made that the force-transmitting mechanism causes a dorsiflexion of the foot part in the event of a knee flexion over a first knee flexion angle range and causes a plantar flexion of the foot part in the event of further knee flexion after a knee flexion limit angle has been exceeded. When a user of an orthopedic device sits down, the thigh part moves about the knee joint axis. The center of gravity of the body is likewise pivoted about the knee joint axis in the context of a circular movement, which has the effect that the center of gravity of the body is moved very quickly out from the region of the support surface of the foot part. The center of gravity of the body then lies behind the support surface, which has the effect that the whole body tilts rearward. A user of an orthopedic device has to compensate for this by way of a sound leg or with the aid of supporting devices or the arms. By means of a constrained dorsiflexion of the foot part, a forward rotation of the lower-leg part about the ankle joint axis takes place, such that the knee joint axis is moved forward. This movement has the effect that the center of gravity of the body is shifted forward under the support surface of the foot part, over a certain knee flexion range, such that a tendency to tilt toward the rear is suppressed or at least reduced. After a knee flexion limit angle, which can be set, has been exceeded, further bending of the knee causes a plantar flexion of the foot part. During the process of sitting down, this plantar flexion further guides the foot part such that the pivoting movement of the lower-leg part relative to the foot part is in the opposite direction, i.e. in a rearward direction, as a result of which the center of gravity of the body is shifted farther to the rear on account of the pivoting movement of the knee joint axis.

In addition, the plantar flexion helps the patient to sit down in such a way that he comes into contact with the seat surface at the desired position and does not land on the front edge of the seat surface. During the knee flexion when sitting down, two mutually opposite movements are performed in succession by the lower-leg part about the ankle joint axis as the knee flexion increases, i.e. as the enclosed angle between the rear face of the thigh in the direction of walking and the rear face of the lower leg decreases, with a movement reversal when a knee flexion limit angle is reached. First of all, a pivoting of the lower-leg part about the ankle joint axis takes place in the walking direction, i.e. in a forward direction, as a result of which the knee joint axis is shifted forward in the walking direction. After the knee flexion limit angle has been reached, the pivoting movement about the ankle joint axis is reversed, and the knee joint axis and therefore also the center of gravity of the body are shifted counter to the walking direction, i.e. in a rearward direction.

The force-transmitting mechanism can be designed as a hydraulic system or as a mechanical coupling mechanism that transmits tensile force and compressive force. An embodiment as a mechanical coupling mechanism that transmits tensile force and compressive force has the advantage of less outlay in terms of construction and easy retrofitting. Transmission ratios can be easily adapted by changes of length. By means of a hydraulic system with cylinders and pistons, lines and switching valves, force transmission from the knee joint to the foot part can take place easily and in a way that takes up little space. The movement reversal can be performed via a switching valve, which can be actuated mechanically and/or electrically.

In an embodiment of the force-transmitting mechanism as a mechanical coupling mechanism, a first bearing spaced apart from the first pivot axis can be mounted on the foot part, and a second bearing spaced apart from the second pivot axis can be mounted on the thigh part, wherein the first bearing adopts a maximum distal or proximal position when the knee flexion limit angle is reached. By fixing the position of the maximum distal or proximal position of the first bearing when the knee flexion limit angle is reached, the position of the movement reversal of the coupling mechanism is defined. Depending on the arrangement of the mechanical coupling mechanism on the foot part, i.e. in front of or behind the ankle joint axis in the walking direction, different movements are brought about by a pivoting about the knee joint axis. The first bearing executes a circular movement about the knee joint axis and, at the fixed knee flexion limit angle, reaches the maximum or distal vertex of the trajectory. Upon continued flexion of the knee, this leads to a movement reversal either in the direction of the ankle joint axis or away from the latter, such that a plantar flexion is performed after the knee flexion limit angle has been reached. If, for example in a starting position in which the knee angle is maximal, the second bearing lies in front of the knee joint axis in the walking direction, the first bearing on the foot part is likewise situated in front of the ankle joint axis in the walking direction, such that the second bearing is at a maximum proximal position when the knee flexion limit angle is reached. Accordingly, the second bearing is at a maximum distance from the ankle joint axis, and the ankle joint angle or plantar flexion angle is minimal. Then, upon further bending of the knee, the flexion angle is further reduced and, on account of the circular movement or approximate circular movement of the second bearing, the coupling mechanism is shifted again in the direction of the ankle joint axis, which leads to plantar flexion. Accordingly, when the maximum knee flexion angle is reached, the first bearing is also at a maximum proximal position, such that a maximum dorsiflexion occurs which, upon further bending, is converted in reverse to a plantar flexion. When the second bearing is located behind the knee joint axis in the extended position of the knee joint, the first bearing is likewise arranged behind the ankle joint axis, such that both bearings are located behind the connecting line between the knee joint axis and the ankle joint axis. When the second bearing is brought to a maximum distal position by the knee flexion movement, the first bearing is also located in a maximum distal position, and the foot part in a position of maximum dorsiflexion. The second bearing adopts a maximum distal position when it lies on the connecting line between the knee joint axis and the ankle joint axis; the second bearing adopts a maximum proximal position when it is located, proximally of the knee joint axis, on the connecting line between the ankle joint axis and the knee joint axis.

In a development of the invention, the position of at least one of the bearings is adjustable in order to adjust the extent of the dorsiflexion or plantar flexion, i.e. in order to be able to adjust the lever ratios. In addition, the position of the bearings can be adjusted in terms of their angle setting, for example in order to adjust the knee flexion limit angle. For example, if the second bearing is rotatable about the knee joint axis and can be fixed in a defined, selectable position, it is thus possible to adjust the knee flexion limit angle, i.e. the angle starting from which a forward shift of the knee joint axis is reversed to a rearward shift. An adjustment can also be made by changing the length of the force-transmitting mechanism.

The bearings can be guided on a circular trajectory. Alternatively to this, it is possible to provide a slotted guide for the bearings, such that it is possible to assign a knee angle profile to an ankle angle profile in almost any desired way.

The bearings can be secured detachably on the foot part and/or the thigh part, so as to be able to retrofit existing prosthetic knee joints or orthotic knee joints with foot parts attached thereto. This is easily possible in a mechanical embodiment of the force-transmitting mechanism. If one bearing for a coupling element is already arranged or formed on a foot part or a thigh part, the still missing bearing can be retrofitted individually, such that a device according to the invention can be produced from an already existing orthopedic device without an aid to sitting down and standing up.

The length of the coupling mechanism between the bearings is adjustable, in order to be able to carry out an individual adaptation to the particular patient.

The knee flexion limit angle preferably lies in a range of between 50° and 80°, in particular in a range of between 60° and 80°, in particular at 75°.

In a development of the invention, provision is made that an energy store and/or a damper mechanism are arranged between the lower-leg part and the thigh part. It is thereby possible, on the one hand, to damp the movement when sitting down, in order to prevent the body from going down too quickly. On the other hand, a device with an energy store, for example a spring, provides assistance in standing up, thus making it easier for a patient to stand up. If the energy store is charged during the process of sitting down, the energy can be released by a movement reversal, so as to deliver assistance in standing up.

In order to control the bending of the knee when sitting down, a damping element is arranged in the orthopedic device in one embodiment of the invention. Assistance is thus given for controlled lowering of the center of gravity of the body as the knee flexion increases. In a development of the invention, the damping element can be designed as a progressive damping element which, at an increasing knee flexion angle, i.e. in the event of increasing knee flexion, has a progressive increase in the generated force. As the knee flexion increases, the damping force applied by the damping element thus increases. The degree of the progression of the damping force can be provided either via a mechanical design of the damper mechanism, in which design the contours of the piston and/or of the cylinder and/or bypasses at an increasing flexion angle increase the flow resistance, or via a mechatronic actuation of a valve, e.g. a control valve or an adjustable throttle valve, for changing the hydraulic resistance. The damper mechanism here has a comparatively low initial resistance in the event of a knee joint at maximum extension and rises to a very high resistance at a knee angle flexion range of between 70° and 90°. The progression is preferably stepless. The level of damping is adjustable, such that the device can be adapted to patients of different weights. The adaptation and adjustment of the level of damping can take place via a manually adjustable valve or a throttle or by programming of a mechatronically actuated valve.

The damper element can also be used to completely block the knee joint against unwanted bending of the knee joint during walking or standing, in order to prevent unwanted or uncontrolled bending of the knee joint. In the case of a hydraulic damper element, a crossflow from an extension chamber into a flexion chamber or vice versa is blocked, such that knee flexion is completely prevented. The switching off of the blocking of the knee flexion, and the then associated decrease in the bending resistance, or a reduced flexion damping for sitting down, can be effected either by a manual switch or a mechatronic detection of the process of sitting down, for example via a movement-based control system, or by a control system which, by way of load sensors, detects when a sitting-down movement takes place.

Standing up can likewise be assisted by a hydraulically implemented blocking process with a movement reversal, similar to a ratchet mechanism of a mechanical solution, by means of a renewed knee flexion being blocked after the extension movement has been interrupted during standing up. It is thus possible for a user of the orthopedic device to shift a load onto a flexed prosthesis or orthosis and thus perform the process of standing up in several stages. Starting from a flexion angle of 20° to 30°, i.e. a remaining extension angle of 20° to 30° as far as the position of maximum extension, this hydraulic ratchet mechanism can be deactivated again. The mechanism or the switch can be realized either mechanically via the design of the hydraulics or mechatronically via a movement-dependent switching of a valve on the basis of sensor data. The hydraulics can further be configured such that an increase of the extension damping is present before the mechanical extension stop is reached, i.e. before the mechanically predefined maximum extension is reached, in order to damp a hard impact at the extension stop when standing up, so as to enhance patient comfort. Damping of an extension stop can be implemented either mechanically via a piston geometry or an elastomer element or mechatronically via an angle-dependent actuation of a valve.

After the knee flexion angle usually required for sitting has been reached, the flexion damping can be reduced, if appropriate canceled, in order to permit free swinging of the lower leg or the lower part after lifting of the prosthesis or orthosis. Thus, the patient or the user of the orthopedic device can easily bring the lower leg to the desired position when sitting.

The respective joint device can be assigned an energy store, for example in order to store energy when sitting down and to release this energy again to assist the standing-up movement. Moreover, the energy store can be assigned a catch which prevents the stored energy from being released at the wrong time, e.g. when seated. This catch can be opened either manually or via a sensor-controlled actuator, in order to assist the standing-up process at a desired time. It is thereby possible for energy, once stored, to be released when standing up or for standing up, in order to assist in a standing-up movement.

The ankle joint and/or the knee joint can be assigned an actuatable blocking mechanism which prevents bending of the joint and which permits safe walking with a stiff leg. For sitting down, this catch is unlocked manually or via sensors and an actuator. A locking device, which blocks a flexion of the knee joint, permits walking with a prosthetic leg, without the risk of bending or buckling.

Illustrative embodiments of the invention are explained in more detail below with reference to the attached figures, in which:

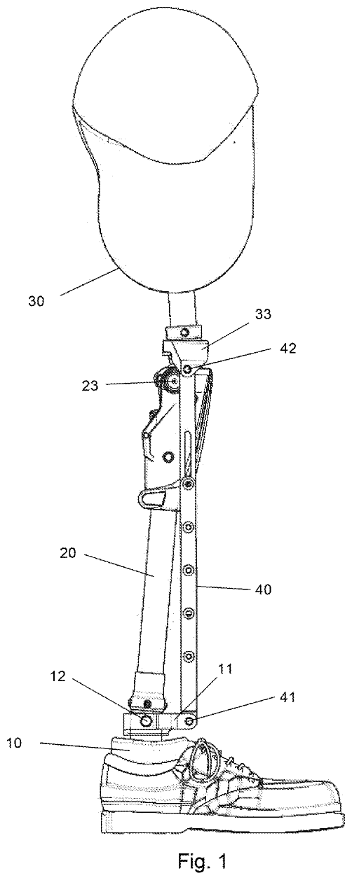

FIG. 1 shows a side view of an orthopedic device in the form of a prosthesis, in an extended position;

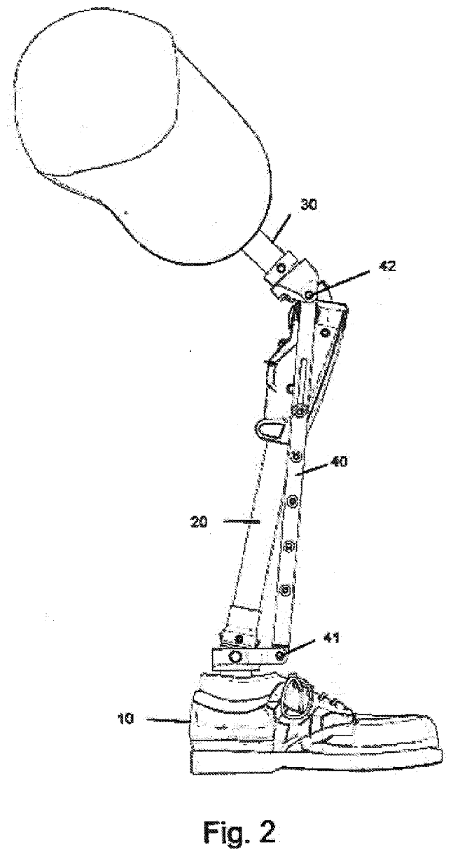

FIG. 2 shows a view in a flexed position at the knee flexion limit angle;

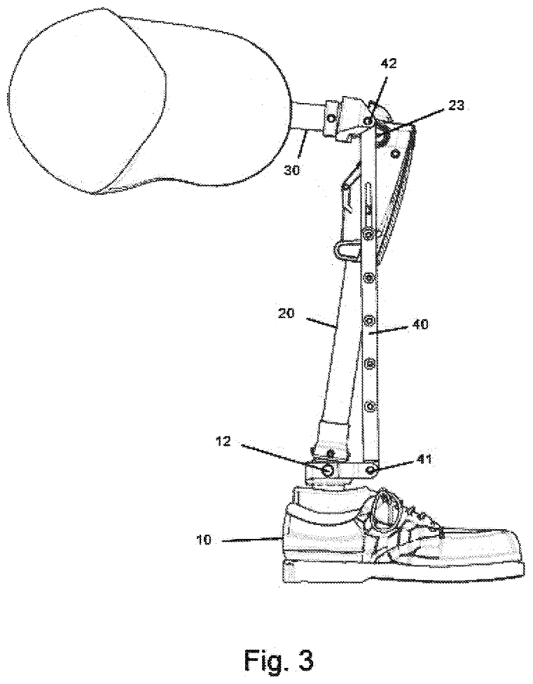

FIG. 3 shows a side view in a sitting position;

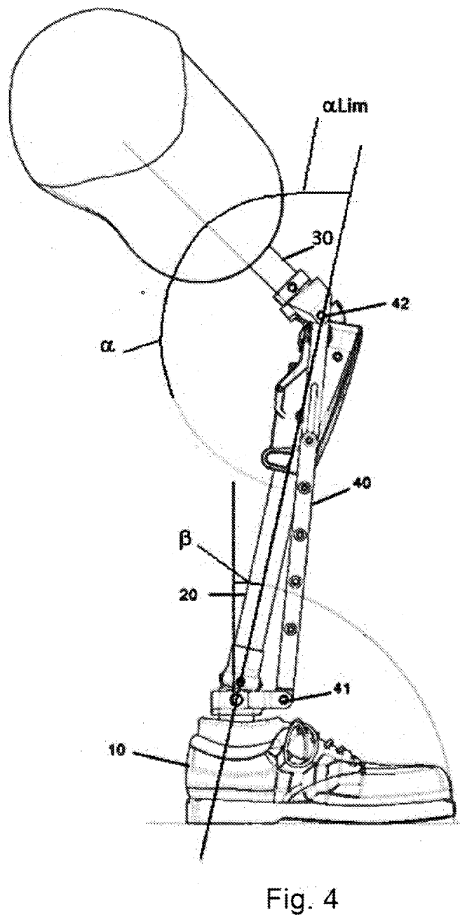

FIG. 4 shows a view according to FIG. 2, with angle indications;

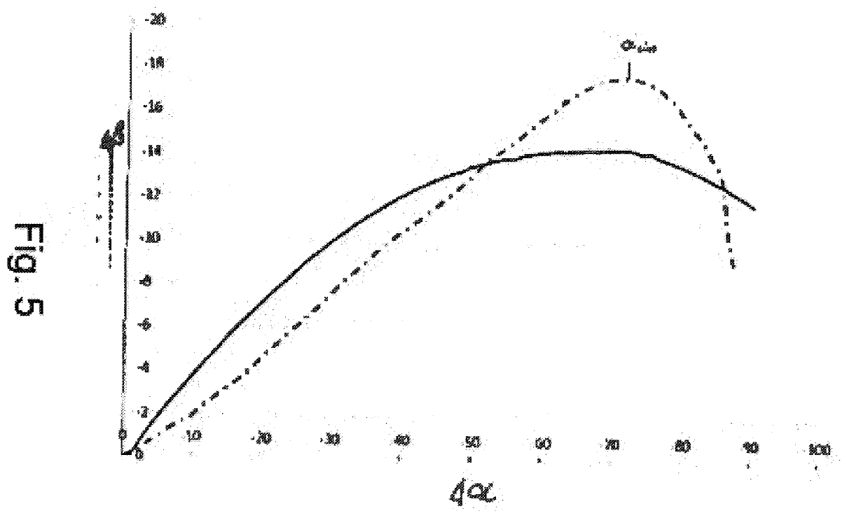

FIG. 5 shows a diagram of the ankle angle over the knee angle;

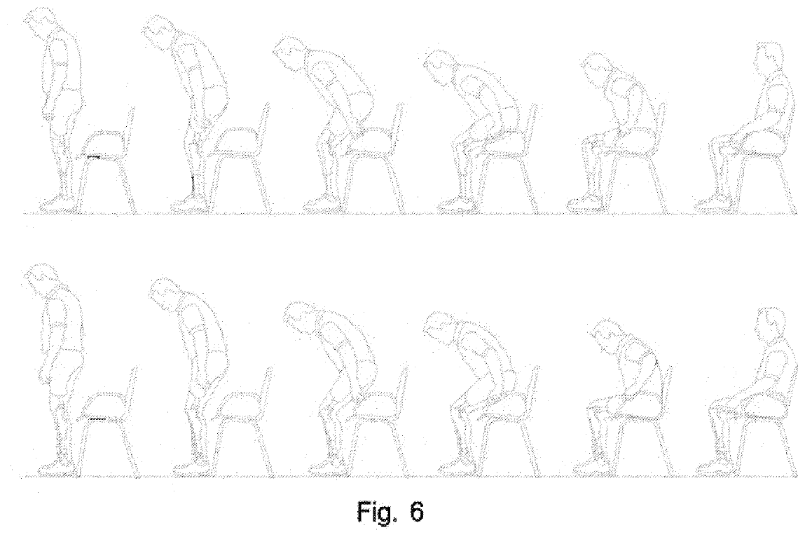

FIG. 6 shows two sets of views of a sitting-down process;

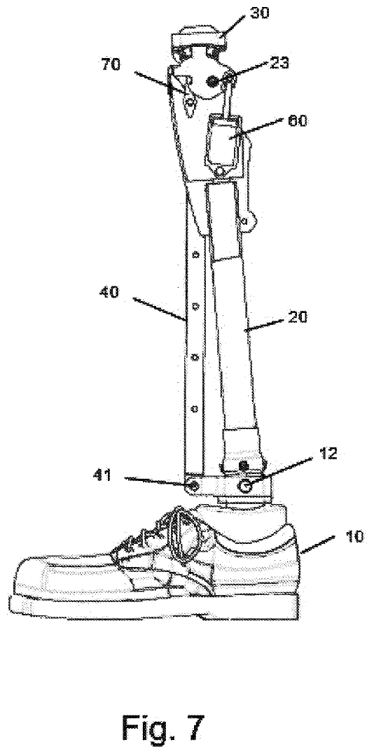

FIG. 7 shows a variant in partial cross section, with a damper and blocking mechanism;

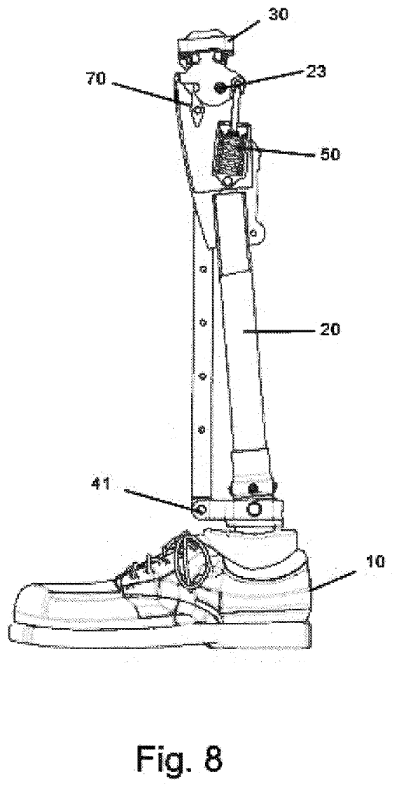

FIG. 8 shows a variant of the device with an energy store;

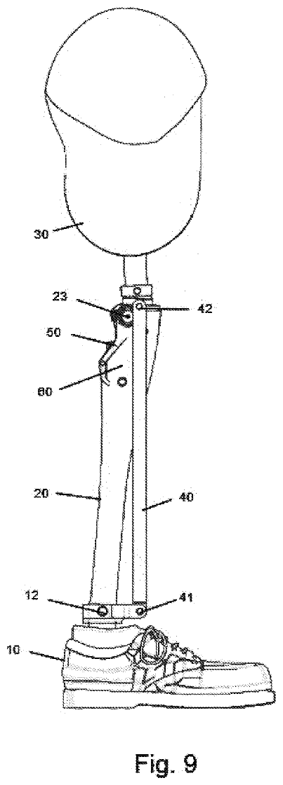

FIG. 9 shows a further variant of the invention; and

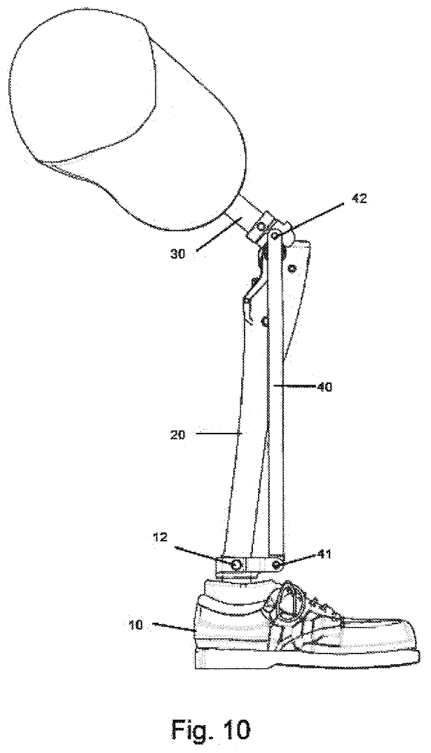

FIG. 10 shows a variant of FIG. 9 in a flexed position at the knee flexion limit angle;

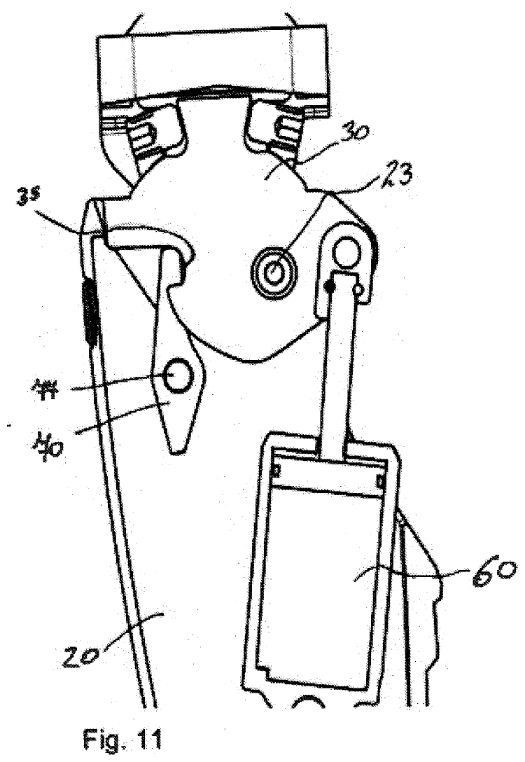

FIG. 11 shows a detail of FIG. 7;

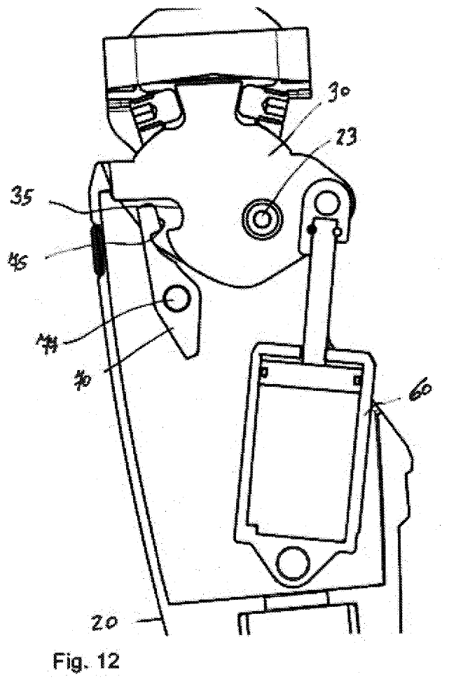

FIG. 12 shows a variant of the invention according to FIG. 11, in an opened position, and

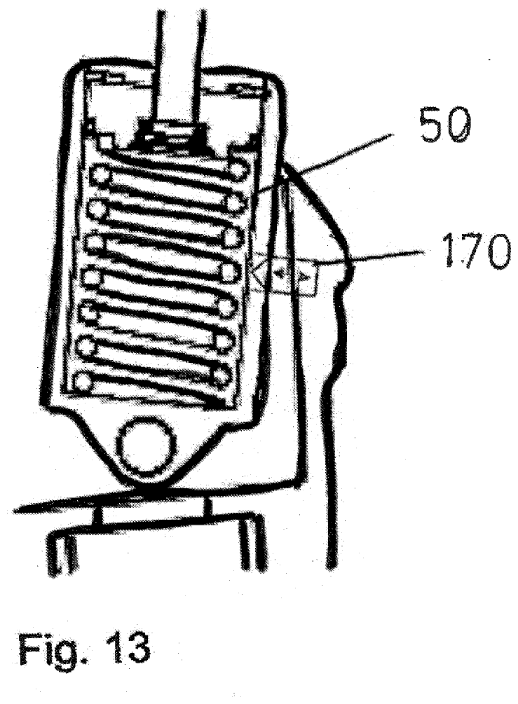

FIG. 13 shows a variant of the invention, with a locking mechanism on an energy store;

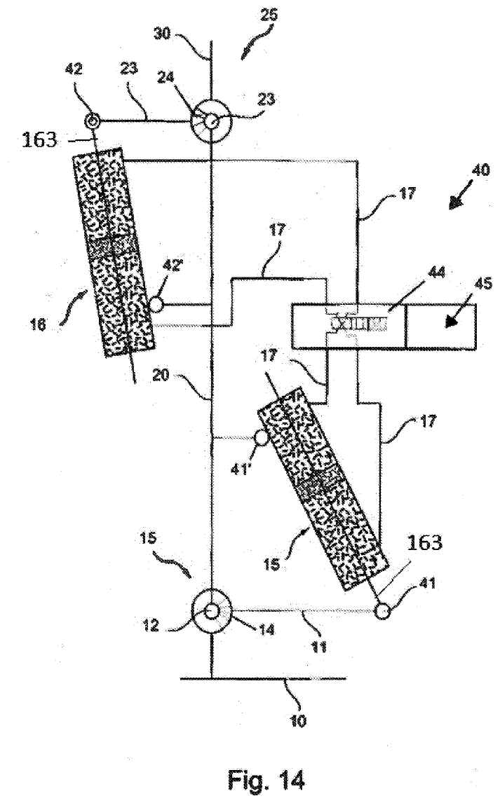

FIG. 14 shows a schematic view of a hydraulic system for force transmission;

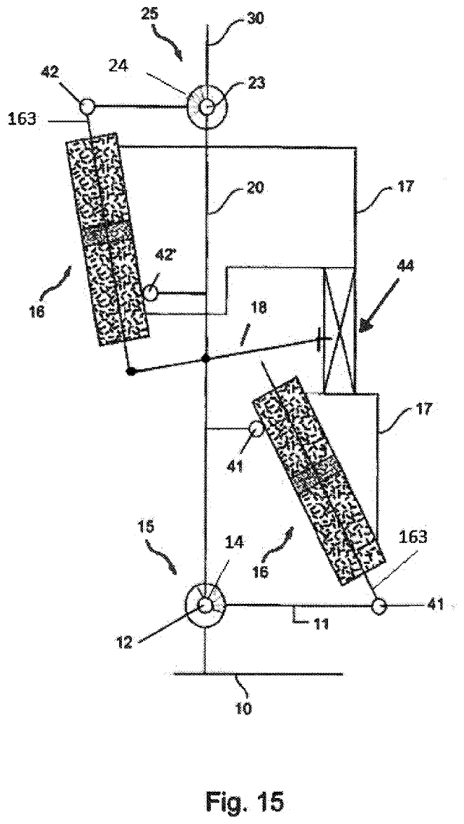

FIG. 15 shows a variant of FIG. 14; and

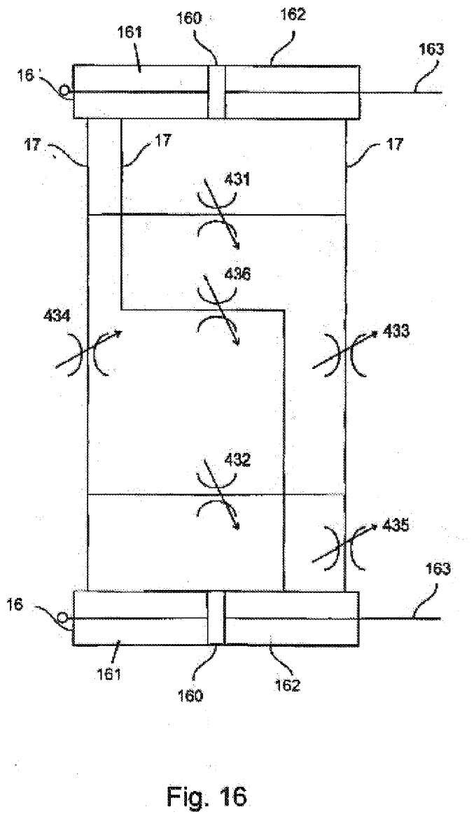

FIG. 16 shows a schematic view of a variant of a hydraulic system.

FIG. 1 shows a side view of an orthopedic device in the form of a prosthesis for a lower limb, having a foot part 10, a lower-leg part 20 and a thigh part 30. The thigh part 30 is designed as a prosthesis socket with an attached tube piece for connection to an upper part of a prosthetic knee joint. The lower-leg part 20 has a lower-leg tube and lower part of a prosthetic knee joint and is connected to the thigh part 30 pivotably about a second pivot axis 23. The foot part 10, with a prosthetic foot inserted in a shoe, is connected to the lower-leg part 20 pivotably about a first pivot axis 12. The orthopedic device is located in the state of maximum extension. A first jib 11 is secured rigidly on the foot part 10. The first pivot axis 12 extends inside the jib 11, such that the lower-leg part 20 can pivot relative to the foot part 10 about the first pivot axis 12 when the foot part 10 is placed flat on the ground. The first jib 11 can be designed to be secured detachably on the foot part 10 and fixed thereon. It is thereby possible also to arrange the jib 11 at a later stage on a finished orthopedic device, for example a prosthesis or an orthosis. In the illustrative embodiment shown, a first bearing 41 is formed on the first jib 11, spaced apart in the frontal direction from the first pivot axis 12, which first bearing 41 lies forward of the pivot axis 12 in the normal walking direction. A force-transmitting mechanism 40 in the form of a mechanical coupling element is arranged at the first bearing 41 and extends in a proximal direction. The coupling element 40 is adjustable in length. For this purpose, the coupling element 40 is configured in two parts and has, in the proximal part, a longitudinal guide bore along which the distal part can be displaced. By way of screws, the first part can then be fixed in the respectively desired position on the second part. The proximal end of the coupling element 40 is secured at a second bearing 42, which is formed on a second jib 33 that is secured on the upper part 30. The second jib 33 can likewise be secured detachably on the thigh part 30 or the upper part of the prosthetic knee joint in order to permit retrofitting. In the illustrated position of extension of the orthopedic device, the second bearing 42 lies forward of the second pivot axis 23 in the walking direction, such that both bearings 41, 42 lie forward of the connecting line between the two pivot axes 12, 23 on the ankle joint and the knee joint. The second bearing 42 is located closer to the second pivot axis 23 than the first bearing 41 is to the first pivot axis 12. Gearing can take place via the length ratio, i.e. via the distances of the bearings 41, 42 to the respective pivot axes 12, 23. The shorter the distance of the second bearing 42 to the second pivot axis 23, the shorter the path traveled on the circular trajectory, and the smaller the pivoting angle of the lower-leg part 12 relative to the static foot part 10. The first bearing 41 lies on a common plane with the first pivot axis 12, which runs substantially parallel to a flat ground surface; the second bearing 42 is positioned posteriorly with respect to the second pivot axis 23, i.e. pivoted counterclockwise by an angle relative to the horizontal. When the thigh part 30 is pivoted counterclockwise about the second pivot axis 23, the enclosed angle between the rear face of the thigh part 30 and the rear face of the lower-leg part 20 decreases, this angle also being called the knee angle. Such a pivoting movement is a knee flexion. The second bearing 42 executes a movement on a circular trajectory about the second pivot axis 23, as a result of which, in addition to a horizontal component, there is a vertical component in the movement of the second bearing 42. On account of the rigid coupling and the constant distance between the first bearing 41 and the second bearing 42, a pivoting movement of the foot part takes place counterclockwise about the first pivot axis 12. A lifting of the top surface or dorsum of the foot in the direction of the lower-leg part 30, i.e. the movement of the foot tip about the pivot axis 12 in the direction of the lower-leg part 20, is called dorsiflexion, while a reverse movement in which the sole or tip of the foot is moved in the direction of the ground is called plantar flexion. During plantar flexion, the angle between the top of the foot part 10 and the front face of the lower-leg part 20 increases, whereas the angle decreases during dorsiflexion.

In FIG. 2, the embodiment of the prosthesis according to FIG. 1 is shown in a flexed position in which the thigh part 30 has adopted the knee flexion limit angle. Arranged between the lower-leg part 20 and the thigh part 30 is a prosthetic knee joint which, for example, can also have an energy store and/or a damper mechanism. In the position in FIG. 2, the force-transmitting mechanism 40 is flush with the connecting line between the upper bearing 42 and the pivot axis 23, and the maximum forward rotation of the lower-leg part 20 is reached.

In FIG. 3, the orthopedic device according to FIG. 2 is shown in a further flexed position, which is to say that the thigh part 30 has been pivoted farther in the direction of the lower-leg part 20 counterclockwise about the second pivot axis 23. The second bearing 42 has been moved farther counterclockwise on the circular trajectory about the second pivot axis 23. On account of the movement component of the circular trajectory directed toward the foot part 10, a compressive force is exerted on the coupling element 40 such that, in the event of a knee flexion beyond the knee flexion limit angle shown in the position according to FIG. 2, a force direction reversal and thus also a movement reversal of the pivoting movement of the lower part 20 relative to the foot part 10 about the first pivot axis 12 takes place. In the event of the knee flexion increasing after the knee flexion limit angle has been reached, the lower-leg part 20 is pivoted counterclockwise about the first pivot axis 12. The foot part 10 remains on the ground, and the knee joint axis 23 describes a curve about the first pivot axis 12 and, on account of the comparatively large distance of the pivot axes 12, 23 from each other, is shifted substantially rearward in the horizontal direction, i.e. counter to the walking direction. After a maximum forward position has been reached at the knee flexion limit angle, the second pivot axis 23 migrates rearward again. On account of the comparatively small distal movement path after the vertex has been reached at the knee flexion limit angle, this movement is comparatively short, with the result that, in the illustrated position which substantially corresponds to a seated position, the knee joint axis 23, with the foot part 10 placed flat on the ground, is shifted slightly forward in relation to an extended position of the orthopedic device, such that the lower-leg part 20 slopes gently forward in the seated position.

In FIG. 4, the position according to FIG. 2 is shown with the associated angle relationships. The ankle angle β is plotted between the vertical or perpendicular, running through the pivot axis 12, and the connecting line between the pivot axes 12, 23, which are explained in FIG. 1, wherein the view from the perpendicular through the pivot axis 12 between the foot part 10 and the lower-leg part 20 is plotted on the shortest path to the lower-leg part 20. The knee angle α is plotted between the connecting line of the pivot axes 12, 23 and the longitudinal extent of the thigh part 30 on the rear face; the knee flexion limit angle α_{Lim} is the adjacent angle of the knee angle α and yields 180° together with the latter. In the position in FIG. 4, the knee flexion limit angle α_{Lim} is reached when a maximum ankle angle β is reached. The second bearing 42 is located at a maximum distance from the first pivot axis 12.

FIG. 5 shows two angle profiles, one for a sound leg and one for an orthopedic device according to the present invention, wherein the profile for the orthopedic device is shown by a solid line, and the profile for a sound leg is shown by a broken line. The angle changes Δβ of the ankle joint angle, starting from the minimum ankle joint angle β at the position of maximum extension of the knee joint, are plotted on the ordinate. The abscissa shows the change of the knee angle α starting from a position of maximum extension in which the knee angle α measures approximately 180°. It will be seen from FIG. 5 that the ankle joint angle β increases during sitting down and standing up from the maximum knee angle α as far as a knee angle limit value. The knee angle change Δα shows that, with a decreasing knee angle α over a range of ca. 75°, i.e. with a flexion of approximately 75°, starting from the extended position, a knee flexion limit angle α_{Lim} is reached. At this knee flexion limit angle α_{Lim}, the ankle joint angle β is at a maximum, which is to say that the ankle joint change Δβ is at a maximum. In the illustrative embodiment shown in connection with FIG. 4, the lower-leg part 20 is pivoted forward, i.e. in the walking direction, by approximately 18° from a starting position. Upon further flexion of the knee joint as far as an approximately horizontal position of the thigh part 30, at which the knee angle α has reduced by ca. 90°, a return pivoting of the lower-leg part 20 takes place when sitting down or standing up, i.e. a shifting of the lower-leg part 20 in the rearward direction about the first pivot axis 12, as far as a pivoting angle of 5° to 10° for the ankle joint angle β, starting from the starting position. That means that, in the usual seated position, the natural ankle joint executes a dorsiflexion of 5° to 10° when the foot is not moved when sitting down. By contrast, in the solid line, the profile of the change of the ankle joint angle β is shown over the change of the knee joint angle α. The profile corresponds qualitatively to that of the sound leg, as is shown in the version indicated by a broken line. The somewhat different, flatter profile of the curve for the orthopedic device can be supplemented by a deformation of the foot or of the foot part of the orthopedic device, resulting in an approximation to or even complete similarity to the natural profile. In the orthopedic device, a maximum dorsiflexion is also achieved at a knee flexion limit angle α_{Lim} which corresponds substantially to the knee flexion limit angle of the sound leg. In the illustrative embodiment shown, the dorsiflexion is 14°. Subsequent to the knee flexion limit angle α_{Lim}, the ankle joint angle β is reduced again upon further bending, i.e. the lower-leg part 20 is pivoted again in a reverse direction about the first pivot axis 12, such that a more or less natural position of the lower-leg part 20 and of the knee joint, and therefore also of the thigh part 30, can be achieved during sitting down and also during standing up.

Two different sequences of sitting down are shown one above the other in FIG. 6. The images at the top show the six phases of sitting down, from the standing position to the seated position, without the orthopedic device according to the invention, while the lower sequence shows these phases with the device according to the invention. From left to right, starting from the position of maximum extension in the knee joint, bending of the knee takes place, where the lower-leg part 20 remains substantially perpendicular, with the foot part placed flat on the ground. The center of gravity of the patient's body is moved in a circular trajectory about the knee joint axis and, already in the third movement phase, leaves the support surface provided by the feet. In this way, the whole body tilts toward the rear, and the user of the orthopedic device has to support himself with his hands. This supporting phase is shown in the fourth image from the left, where the thigh has approximately an angle of 70°. The whole weight has to be taken up by the intact leg and the arms. The user of the orthopedic device can then drop into the chair and sit straight up again.

The six phases are shown correspondingly in the lower images. In the second image from the left, it will be seen that the lower-leg part already pivots forward upon slight knee flexion of the orthopedic device, such that the center of gravity remains above the support surface of the feet. In the third movement phase, the ankle joint angle β is further reduced, the knee joint axis is moved farther forward, and the center of gravity of the body lies farther to the front, in the region of the support surface of the feet, compared to an uncoupled movement between knee flexion and dorsiflexion. Sitting down in the fourth movement phase is made considerably easier; the user does not drop with his pelvis into the backrest, and instead he sits down considerably farther forward on the seat surface. In the fully lowered position, the lower-leg part 20 is located in a slightly inclined position, which substantially corresponds to a natural position of a lower leg.

A variant of the invention is shown in FIG. 7, in which a damper mechanism 60 is arranged in the lower-leg part 20. By means of a piston rod being mounted at a distance from the second pivot axis 23, a pivoting movement about the pivot axis 23 is converted into a linear movement of a cylinder into a piston. An energy store can also be arranged inside the damper mechanism 60, resulting in a combination of damper mechanism and energy store.

A sectional view of a variant according to FIG. 7 is shown in FIG. 8, from which it can be seen that the lower-leg part 20 is of modular design and is composed of a lower-leg tube, and of a joint lower part in which the energy store 50 is arranged. The energy store 50, in the form of a spring, is charged via a knee flexion in which the upper part or thigh part 30 moves in the flexion direction about the second pivot axis 23. To the rear of the second pivot axis 23, i.e. posterior to the pivot axis 23, a bolt is fitted which can convert a pivoting movement about the pivot axis 23 into a linear movement for compression of the energy store 50. The energy store 50 can be equipped with a locking device in order to selectively output the stored energy again to the bolt, so as to permit or assist an extension movement.

In the illustrative embodiment shown in FIGS. 7 and 8, the knee joint is assigned a locking mechanism 70 in the form of a pawl which locks the knee joint in the extended position against flexion about the pivot axis 23. The locking mechanism 70 is designed to be actuated manually or by motor and is explained in more detail with reference to FIGS. 11 and 12. The pawl 70 is mounted in the lower part or lower-leg part 20 and engages in a recess in the upper part or thigh part 30 in order to lock the knee joint. In order to permit flexion about the pivot axis 23, the pawl 70 is pivoted and disengaged from the upper part or the thigh part 30.

FIG. 9 shows a variant of the invention in a position according to FIG. 1. In contrast to the embodiment according to FIG. 1, the second bearing 42 in the illustrative embodiment of FIG. 9 is not formed on a separate jib 33 but instead on the thigh part 30 or the upper part of the knee joint. This results in an integrated product that is already fully assembled. The first bearing 12 can be formed on a separate jib 11 or on the foot part 10 itself.

FIG. 10 shows a position of the orthopedic device in which the thigh part 30 has executed a flexion movement about the second pivot axis 23 as far as the knee flexion limit angle. The thigh part 30 has been pivoted counterclockwise; the second bearing 42 is located in a maximum proximal position, i.e. on a vertex of the curve. The foot part 10 is still placed fully on the ground. On account of the rigid coupling between the first bearing 41 and the second bearing 42 and the tensile force transmission, the lower-leg part 20 was pivoted clockwise about the pivot axis 12. The second pivot axis 23 was thereby shifted in the anterior direction, i.e. forward in the normal walking direction, such that the second axis 23 is located farther in front of the first pivot axis 12, approximately level with the first bearing 41 in the position shown. The knee angle in the position shown has reached a knee flexion limit angle from which, upon further flexion, i.e. upon further pivoting of the thigh part 30 counterclockwise about the pivot axis 23, a movement reversal of the lower-leg part 20 is effected about the first pivot axis 12. On account of the distally acting movement component of the circular movement of the second bearing 42, a compressive force is exerted by the second bearing 42 on the first bearing 41 via the mechanical coupling element 40 upon further flexion of the thigh part 30. In this way, the lower-leg part 20 pivots counterclockwise about the first pivot axis 12 upon further pivoting. Instead of the coupling element 40 being secured via a jib 33 that can be secured subsequently on the upper part, the coupling element 40 in the variant according to FIG. 10 is arranged on a bearing 42 which is integrated in the tight part 30 or upper part of the prosthetic knee joint. The prosthesis is designed for the securing of the coupling element 40; the necessary securing sites are taken into account in the design and production of the components.

FIG. 11 shows a detail of FIG. 7 with the prosthetic knee joint which has an upper part or thigh part 35 and the lower part 20, which are mounted pivotably on each other about a pivot axis 23. The prosthetic knee joint is located in an extended position. On the lower part 20, a locking mechanism 70 in the form of a pawl is mounted on a pivot pin 77 so as to be pivotable about a pivot axis. The proximal end of the pawl 70 engages in a recess 35 in the upper part or thigh part 30 and blocks a flexion about the pivot axis 23. In the position shown, the prosthetic knee joint is held via the locking mechanism 70, such that no flexion can occur. Unwanted bending of the prosthetic knee joint cannot take place.

FIG. 12 shows the prosthetic knee joint according to FIG. 11 in an unlocked and enabled extended position. The pawl or the locking mechanism 70 has been shifted counterclockwise about the pin 77, for example by mechanical actuation by cable or by lever, such that the proximal end of the locking mechanism 70 with the projection 75 disengages from the recess 35. As an alternative to a purely manual actuation of the locking mechanism 70, said actuation can take place by motor, for example via a switchable magnet, an electric motor or similar. The actuation can be a sensor-controlled actuation or involve actuation of a switch which can be arranged on the prosthesis or by remote control of an operating element at another location. In the position according to FIG. 12, the prosthetic knee joint is unlocked and can be shifted in the direction of flexion and in the direction of extension.

FIG. 13 shows a variant of the invention according to FIG. 8 where, instead of purely a damper mechanism 60, an energy store 50 is arranged between the upper part and the lower part. The energy store 50 is designed in the form of a compressible spring. The energy store 50 is assigned a locking mechanism 170 which, as is indicated in the arrow direction, can be in form-fit engagement with the energy store 50. For example, if the spring as energy source 50 is compressed and the locking mechanism 170 is driven into the free space and blocks a relaxation of the spring 50, a reverse transmission of the energy is prevented or obstructed, since the spring 50 cannot relax or can only partially relax. It is only after the locking mechanism 170 has been released that the spring 50 is freed and is able to relax in order to deliver the stored energy and assist an extension. The locking mechanism 70 for the knee joint can be coupled with the locking mechanism 170 for the energy store 50 and can be configured for separate actuation on the prosthesis or the orthosis.

FIG. 14 shows a schematic view of an orthopedic device with the foot part 10, the lower-leg part 20 and the thigh part 30, which are pivotably connected to one another via the respective joints, namely via the ankle joint 15 and the knee joint 25. Instead of a mechanical force-transmitting mechanism, a first cylinder/piston unit 16 is arranged on the first jib 11 and is connected to the jib 11 at the first bearing 41 via a piston rod 163. The housing of the cylinder/piston unit 16 is mounted pivotably on a bearing 41′ at the lower leg side. The piston/cylinder unit 16 divides two chambers via the piston. The two chambers are connected fluidically to a valve block 44 via lines 17. In the illustrative embodiment shown, the valve block 44 has a three-way valve which is coupled via an actuator 45 provided with a controller. By way of the actuator 45, it is possible to bring the three-way valve into different positions in order to couple the lines 17 differently to each other. The valve block 44 has output lines 17 which lead to a second piston/cylinder unit 16, the latter being designed corresponding to the first piston/cylinder unit 16. The housing of the second piston/cylinder unit 16 is coupled pivotably to a bearing 42′ at the lower leg side. A piston rod 163 is mounted pivotably at a second bearing 42, which is arranged on a second jib 33. The second piston/cylinder unit 16 is also divided into two chambers by a piston.

Angle sensors 24, 14 are arranged or formed both on the knee joint 25 and on the ankle joint 15 and are coupled to the controller 45 via lines (not shown) or wirelessly. In principle, it is also possible to achieve the desired function and carry out the method via a knee angle sensor 24 alone. Depending on the angle setting of the thigh part 30 relative to the lower-leg part 20, the valve block 44 can be switched on the basis of limit values or threshold values stored in the controller 45. In the illustrated position of the valve block 44, during a knee flexion when the thigh part 30 pivots counterclockwise about the second pivot axis 23, the piston of the piston/cylinder unit 16 connected to the second jib 33 is pressed downward and the volume of the corresponding cylinder chamber is reduced. In this way, hydraulic fluid is conveyed through the line 17 to the valve block 44. In the illustrated valve setting, hydraulic fluid is conveyed from the lower or distal chamber of the upper piston/cylinder unit 16 to the lower or distal chamber of the piston/cylinder unit coupled to the first jib 11. In this way, the piston of the lower piston/cylinder unit 16 is shifted upward or proximally, which leads to a dorsiflexion of the foot part 10. When a knee flexion limit angle is reached and exceeded, the three-way valve is displaced into the valve block 44, such that a parallel coupling of the lines 17 in the valve block 44 takes place. In this way, fluid is conveyed from the lower, distal chamber of the upper piston/cylinder unit 16 into the upper, proximal chamber of the lower, distal piston/cylinder unit 16, which has the effect that the piston rod 163 is pressed out from the distal piston/cylinder unit 16. This pressing out causes a pivoting of the first jib 11 and also of the whole foot part 10 about the first pivot axis 12 and thereby causes a plantar flexion.

In order to decouple a flexion of the thigh part 30 relative to the lower-leg part 20 from the movement of the foot part 10 relative to the lower-leg part 20, the valve block 44 can be shifted to a third position, in which the two piston/cylinder units 16 are fluidically separated from each other. The two chambers respectively separated by a piston are then coupled to each other via a short-circuit line. If adjustable valves are present in the short-circuit lines, an independent adjustment of the damping can then be effected according to sensor values, e.g. of the angle sensors, or else of other sensors such as force sensors, torque sensors, spatial position sensors, acceleration sensors, pressure sensors and/or temperature sensors.

FIG. 15 shows a variant of the orthopedic device with a hydraulic force-transmitting mechanism in which, instead of a sensor-controlled actuation of the valve block 44, a mechanical actuation is provided. By way of a lever 18, which is coupled to a piston rod 163 guided out from the housing of the proximal piston/cylinder unit 16, a mechanical deflection in the valve block 44 is effected via a deflection element, as is shown only schematically. By way of a slide or a rotation mechanism, a corresponding switching of the allocations of the distal and proximal chambers of the respective piston/cylinder units 16 is then performed such that, when a knee flexion angle is reached or exceeded, a dorsiflexion of the foot part 10 is switched to a plantar flexion.

FIG. 16 shows a schematic view of the hydraulic circuit with a plurality of valves 431, 432, 433, 434, 435, 436, which can be actuated individually on the basis of sensor data of the sensors 14, 24, in particular in accordance with the knee angle sensor 24, but also in accordance with one of the abovementioned other sensor types. Both piston/cylinder units 16 have piston rods 163 which protrude from the housing and on which a piston 160 is secured. The piston 160 divides the cylinder into two chambers 161, 162. A protruding end of the piston rod 163 is secured on a jib 11, 33. The other end can either protrude freely from the housing, as a result of which compensation volumes for the hydraulic liquid can be avoided, or end at the piston 160. The hydraulic lines 17 connect the respective distal and proximal cylinder chambers 161, 162 of the two piston/cylinder units 16. A diagonal line 17 connects a distal cylinder chamber 162 to a proximal cylinder chamber 161. At least one valve 431, 432, 433, 434, 435, 436 is arranged in each of the hydraulic lines 17 in order to be able to realize different circuits. For example, if the valves 433, 434, 435 are opened and the remaining valves 431, 432, 436 are closed, this results in a parallel circuit, which has the effect that a shifting of the upper piston 160 to the left leads to a shifting of the lower piston 160 to the right. In order to generate an oppositely directed movement, the two cylinder/piston units 16 have to be routed crossways to each other, for which purpose the valves 431, 434, 435 are closed, while the valves 432, 433, 436 are open.

If the valves 433, 434, 436 are closed, this leads to a decoupling for example of the proximal piston/cylinder unit 16 from the distal piston/cylinder unit 16. By partial closure of the opened valves 431, 432, 435, it is possible to adapt the resistance to shifting.

If the upper valve 431 is now opened, the ankle joint 15 for example remains rigid, whereas the knee joint 25 can be bent. The resistance to bending derives from the hydraulic resistance of the opened valve 431. A stiff knee joint 25 and a movable ankle joint 15 are possible when the valves 432, 435 are opened and the other valves remain closed.

1. An orthopedic device comprising:
a foot part;
a lower-leg part;
a thigh part;
an ankle joint connecting the foot part to the lower-leg part, the foot part being pivotable about a first pivot axis defined by the ankle joint;
a knee joint connecting the lower leg part to the thigh part, the lower leg part being pivotable about a second pivot axis defined by the knee joint;
a force-transmitting mechanism connecting the foot part to the thigh part, the force-transmitting mechanism causing a dorsiflexion of the foot part in the event of a knee flexion over a first knee flexion angle range and causing a plantar flexion of the foot part in the event of further knee flexion after a knee flexion limit angle has been exceeded.

2. The orthopedic device as claimed in claim 1, wherein the force-transmitting mechanism is designed as a hydraulic system or as a mechanical coupling mechanism that transmits tensile force and compressive force.

3. The orthopedic device as claimed in claim 1, wherein the mechanical coupling mechanism is mounted on the foot part at a first bearing spaced apart from the first pivot axis and is mounted on the thigh part at a second bearing spaced apart from the second pivot axis, and the first and second bearings adopt a maximum distal or proximal position when the knee flexion limit angle is reached.

4. The orthopedic device as claimed in claim 3, wherein a position of at least one of the bearings is adjustable.

5. The orthopedic device as claimed in claim 3 wherein the bearings are guided on a circular trajectory.

6. The orthopedic device as claimed in claim 3, wherein the bearings are secured detachably on at least one of the foot part or the thigh part.

7. The orthopedic device as claimed in claim 2, wherein a length of the coupling mechanism is adjustable.

8. The orthopedic device as claimed in claim 1, wherein the knee flexion limit angle is between 50° and 80°.

9. The orthopedic device as claimed in claim 1, wherein at least one of an energy store or a damper mechanism are arranged between at least one of the foot part and the lower leg or between the lower-leg part and the thigh part.

10. The orthopedic device as claimed in claim 1, wherein at least one of the ankle joint or the knee joint are assigned an actuatable locking mechanism.

11. The orthopedic device as claimed in claim 1, wherein the orthopedic device is designed as an orthosis or prosthesis.

12. An orthopedic device comprising:
a lower-leg part;
a thigh part;
a foot part pivotally connected to the lower-leg part and rotatable about a first pivot axis;
a knee joint pivotally coupling the lower leg part to the thigh part, the knee joint defining a second pivot axis;
a force-transmitting mechanism connecting the foot part to the thigh part, the force-transmitting mechanism causing a dorsiflexion of the foot part in the event of a knee flexion over a first knee flexion angle range and causing a plantar flexion of the foot part in the event of further knee flexion after a knee flexion limit angle has been exceeded.

13. The orthopedic device as claimed in claim 12, wherein the force-transmitting mechanism is designed as a hydraulic system or as a mechanical coupling mechanism that transmits tensile force and compressive force.

14. The orthopedic device as claimed in claim 12, wherein the mechanical coupling mechanism is mounted on the foot part at a first bearing spaced apart from the first pivot axis and is mounted on the thigh part at a second bearing spaced apart from the second pivot axis, and the first and second bearings adopt a maximum distal or proximal position when the knee flexion limit angle is reached.

15. The orthopedic device as claimed in claim 14, wherein a position of at least one of the first and second bearings is adjustable.

16. The orthopedic device as claimed in claim 14, wherein the first and second bearings are guided on a circular trajectory.

17. The orthopedic device as claimed in claim 14, wherein the first and second bearings are detachably secured on the foot part or the thigh part.

18. The orthopedic device as claimed in claim 13, wherein the coupling mechanism has an adjustable length.

19. The orthopedic device as claimed in claim 12, wherein the knee flexion limit angle is between 50° and 80°.

20. The orthopedic device as claimed in claim 12, wherein at least one of an energy store or a damper mechanism are arranged between at least one of the foot part and the lower leg or between the lower-leg part and the thigh part.
